(19) United States
(12) Patent Application Publication
(10) Pub. No.: US 2020/0155127 A1
(43) Pub. Date: May 21, 2020

(54) SAMPLE COLLECTION DEVICE

(71) Applicant: Ellume Limited, East Brisbane, Queensland (AU)

(72) Inventors: Scott Robert Fry, East Brisbane, Queensland (AU); Andrea Ranzoni, East Brisbane, Queensland (AU); Sean Andrew Parsons, East Brisbane, Queensland (AU); Mark Gilroy, Bardon, Queensland (AU)

(21) Appl. No.: 16/625,643

(22) PCT Filed: Jul. 12, 2018
(86) PCT No.: PCT/AU2018/050720

§ 371 Date: Dec. 20, 2019

(30) Foreign Application Priority Data
Jul. 13, 2017 (AU) 2017902750

(51) Int. Cl. A61B 10/00; A61F 13/38

(52) U.S. Cl. CPC A61B 10/0096; A61F 13/38; A61B 5/15; A61B 10/0058; A61B 2010/0077

(57) ABSTRACT

A sampling device for collecting and dispensing a sample is disclosed, the sampling device including a sample collector having an absorbent portion configured to collect a sample and a receiving section configured to receive the sample collector after the collection of the sample. In one embodiment, the receiving section includes a dropper, the sample collector configured to be secured to the dropper, the dropper defining an internal chamber in which the absorbent portion of the sample collector is received when the sample collector is secured to the dropper; and a base releasably engaged with the dropper and at least partially housing the dropper, wherein the dropper is configured to be released from the base to allow dispensing of the sample from the dropper. In one embodiment, the absorbent portion interferes with a surface of an internal chamber of the receiving section. In one embodiment, a length adapter for the sample collector is employed and, in another embodiment, a desiccant housing is employed.

### TECHNICAL FIELD

The present disclosure relates to the collection and preparation of samples.

### BACKGROUND

The analysis and testing of a sample, including for detection of constituents of interest in the sample, commonly involves collection of the sample and dispensing of the sample to a test device, a laboratory or other testing facility.

A sample is commonly collected using a swab. Once the sample is collected, the swab can be inserted into a container of fluid, such as buffer solution, so that sample collected by the swab combines with the fluid to form a fluid sample that has one or more properties more suitable for further processing. The swab can then be discarded. A dropper can be used to collect the fluid sample from the container. The fluid sample is dispensed from the dropper onto a suitable region of a test device or other tool to be used in the further processing of the sample. Items used in this process can prove tricky and unwieldy for the user, particularly if the user is not a laboratory professional or in a laboratory environment. The increasing use of point-of-care diagnostics means that it is common for a patient or their caregiver to be directly involved in sample collection and dispensing, and it may therefore be desirable to provide user-friendly, safe and/or reliable sampling apparatus.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

In one aspect, the present disclosure provides a sampling device for collecting and dispensing a sample, the sampling device comprising:

a sample collector having an absorbent portion configured to collect a sample;

a receiving section configured to receive the sample collector after the collection of the sample, the receiving section comprising:
a dropper, the sample collector configured to be secured to the dropper, the dropper defining an internal chamber in which the absorbent portion of the sample collector is received when the sample collector is secured to the dropper; and
a base releasably engaged with the dropper and at least partially housing the dropper, wherein the dropper is configured to be released from the base to allow dispensing of the sample from the dropper.

The dropper may be configured to be released from the base while the sample collector and the dropper are secured together. The dropper may be released from the base by pulling the dropper and base apart in an axial direction of the sampling device. So that the process of releasing the dropper from the base does not cause release of the sample collector from the dropper, a minimum axial force to release the dropper from the base may be lower than a minimum axial force to release the securing of the dropper to the sample collector.

The releasable engagement between the dropper and the base may be via a snap-fitting or by any other means suitable to releasably engage these two parts together such as a friction or interference fit, a clip mechanism, a screw-fitting, a bayonet-fitting or otherwise. The engagement between the dropper and the base need not necessarily be released by pulling in the axial direction. For example, where a screw-fitting is used, engagement may be released by a rotational/unscrewing action.

The sample collector and the dropper may be secured to each other by a screw-fitting or by any other means suitable to secure these two parts together. For example, the securing may also be via a snap-fitting, interference or friction fit, bayonet-fitting or a clip mechanism, etc. However, in some embodiments, the securing may be such that the parts are more securely locked together than the dropper and the base, e.g., to resist separation in an axial direction.

The dropper may comprise a liquid such as a buffer solution in its internal chamber, e.g. prior to securing of the sample collector to the dropper. The liquid may be deposited in the internal chamber during manufacture of the sampling device. A releasable seal may be located over an opening of the dropper to seal the liquid in the internal chamber after manufacture. Alternatively, a user may deposit the liquid into the internal chamber prior to receipt of the sample collector. The user may deposit the liquid from a container such as a vial or ampoule. The container may be packaged with the sampling device. Additionally or alternatively, reagents may be located in the internal chamber. For example, reagents may be coated on or in a surface of the internal chamber or other item located in the internal chamber, e.g. prior to securing of the sample collector to the dropper. Alternatively, a user may deposit the reagents into the internal chamber. The reagents may comprise particles, e.g. nanoparticles, fluorescent particles, quantum dots, etc., that are configured to label a target analyte in the sample for the purpose of performing a subsequent lateral flow test, for example. The reagents may be presented in a liquid, powder, gel, freeze-dried form or otherwise.

When the sample collector is secured to the dropper, sample located on the absorbent portion of the sample collector may combine or react with the liquid and/or reagents to provide a more fluid version of the sample (described herein as a “fluid sample”) and/or a treated version of the sample.

The dropper may include an aperture to dispense the sample from the internal chamber. The aperture may be provided in the dropper during manufacture or may be formed by the user, e.g. immediately prior to dispense of the sample. The dropper may comprise an upper section and a lower section, wherein the upper section has an opening for the sample collector to access the internal chamber of the dropper and the lower section has the aperture to dispense the sample from the dropper. In some embodiments, more than one aperture may be provided to dispense the sample from the dropper. The dropper may include flexible side walls. The flexible side walls may form at least the lower section of the dropper. The flexible side walls may be engageable by a user of the device, e.g. by a user's thumb and forefinger, to press the flexible side walls to cause dispensing of the sample from the internal chamber.

The dropper may include a collar located between the upper section and the lower section of the dropper. The collar may extend radially outwardly of at least the lower section of the dropper and preferably both the upper and lower sections, to contact an inner surface of the base to releasably engage the dropper with the base.

When the dropper is releasably engaged with the base, the lower section of the dropper may be located inside the base and the upper section of the dropper may protrude from the base. The upper section of the dropper may comprise a screw thread for screwing to a complimentary screw thread of the sample collector. The sample collector may comprise a cap configured to close, e.g. seal, by itself or in combination with additional features of the sample collector, the opening of the dropper when the sample collector is secured to the dropper. The complimentary screw thread may be located on a surface of the cap, e.g. an inner surface of the cap.

The sample collector may comprise a swab, the swab providing the absorbent portion of the sample collector. The swab may comprise a rod and the absorbent portion may be located at an end of the rod. The sample collector may comprise an elongate body to which the absorbent portion, and optionally the swab comprising the absorbent portion, may be attached. The elongate body may project from a bottom surface of the cap. The elongate body may be centrally located and may taper from a proximal end connected to the cap towards a distal end where the absorbent portion may be located. The swab may be mounted at the distal end of the elongate body, e.g. by the rod of the swab extending through an opening at the distal end of the elongate body. The elongate body may project, through the opening of the dropper, into the internal chamber of the dropper when the sample collector is received in the internal chamber of the dropper. The elongate body, adjacent its proximal end, may have a diameter that is similar or identical to the opening of the dropper and may therefore substantially close or seal the opening of the dropper as the elongate body extends into the internal chamber. Accordingly, the closing or sealing (e.g., air-tight sealing) of the opening of the dropper may be effected by the cap and/or the elongate body extending from the cap.

The base may include a cup. The cup may have a bottom surface to support the sampling device on a surface and a top opening through which the dropper can enter at least partly into the base. In general, the base may be adapted to rest on a surface to support and/or protect components of the sampling device, such as the dropper, during use.

The one or more apertures of the dropper may be sealed to prevent or substantially resist leakage therefrom before a desired dispensing of the sample from the dropper is conducted. The seal may be provided by a sealing surface that extends across and/or into the aperture. The sealing surface may be connected to, or integrated with, the dropper or the base.

In one embodiment, the base may provide the sealing surface for sealing the aperture of the dropper. For example, the base may comprise a sealing surface to seal the aperture of the dropper when the base is releasably engaged with the dropper and the sealing of the aperture may be released upon releasing of the dropper from the base. Adjacent the aperture, the dropper may have a contoured outer surface and the sealing surface may be configured to contact and conform to the contoured outer surface of the dropper. Additionally or alternatively, the sealing surface may comprise a projection that covers or projects into the aperture of the dropper. In some embodiments, the base may comprise an insert that is located in the cup, the insert comprising the sealing surface. The insert may be pre-shaped so that it includes surfaces that conform to outer surfaces of the dropper to assist with the sealing. Additionally or alternatively, the insert may be a deformable piece of material that conforms to outer surfaces of the dropper to assist with the sealing, e.g. upon the dropper pressing against the insert.

In some embodiments, the sealing surface may be provided by a plug that is at least partially inserted into the aperture to seal the aperture, which plug may remain in place upon release of the dropper from the base and may be removed, e g manually, when releasing of the seal is desired. The plug may be attached to the dropper, e.g. via a hinge or a frangible element.

In one aspect, there is provided a method of collecting a sample and dispensing a portion of the sample to a test device, the method comprising:

using an absorbent portion of a sample collector to collect a sample;

after collecting the sample, securing the sample collector to a dropper of a receiving section such that the absorbent portion is received in an internal chamber of the dropper, the receiving section comprising the dropper and a base that is releasably engaged with, and that at least partially houses, the dropper;

after securing the sample collector to the dropper, releasing the dropper from engagement with the base; and

after releasing the dropper from engagement with the base, dispensing a portion of the sample from the dropper.

The method may use the sampling device as described in the preceding aspect.

The releasing of the dropper from the base may be carried out while the dropper and sample collector are secured together. Similarly, the dispensing of a portion of the sample from the aperture may be carried out while the dropper and sample collector are secured together.

The method may comprise depositing a liquid and/or reagents into the internal chamber of the dropper prior to securing of the sample collector to the dropper. The liquid and/or reagents may be deposited in the internal chamber during manufacture of the sampling device. A releasable sealing layer being located, for example, over an opening of the dropper to seal the liquid and/or reagents in the internal chamber after manufacture. Alternatively, a user may deposit the liquid and/or reagents into the internal chamber. The user may deposit the liquid and/or reagents from one or more containers such as vials or ampoules. The container(s) may be packaged with the sampling device.

In one embodiment, an intra-nasal sample is received from a subject by extending the absorbent portion of the sample collector into a nasal cavity of the subject.

As the sample collector is secured to the dropper of the receiving section the sample located on the absorbent portion may mix or form a solute with the liquid contained in the dropper. The securing technique may assist with this process. For example, the sample collector may be secured to the dropper by screwing. The screwing action may cause rotation of the absorbent portion within the internal chamber. The rotation may assist with extraction of the sample from the absorbent portion and the interaction of the sample with the liquid contained in the dropper, forming a fluid sample in the internal chamber of the dropper. In one embodiment, for example, the rotation of the absorbent portion causes the absorbent portion to be moved against one or more protrusions such as one or more ribs in the internal chamber of the dropper. Frictional and/or shear forces between the absorbent portion and the protrusion(s) may assist with the extraction of the sample.

The sample collector, dropper and base may remain secured/engaged together, for a period of time sufficient to form a desired fluid sample, e.g. through an incubation process or otherwise. As indicated, in some embodiments, reagents may be provided, e.g., deposited, in the dropper for interacting with the sample. For example, capture reagents that are configured to label an analyte of interest, if present in the sample, may be provided in the dropper, e.g. by being coated on an inner surface of the dropper. The period of time in which the sample collector, dropper and base remain secured together may be sufficient for the capture reagents to form labelled complexes with the analyte of interest, e.g. as a precursor to performing immunochromatography using a lateral flow test device to which the fluid sample is subsequently applied.

Once the desired fluid sample has been obtained, the dropper, with fluid sample therein, and while secured to the sample collector, may be released from the base. The release of the dropper from the base may cause unsealing of the aperture of the dropper and may expose flexible sidewalls of the dropper. The user may engage the flexible side walls, e.g. with their thumb and forefinger, to press the flexible side walls to cause dispensing of the sample, via the aperture of the dropper.

In the process of securing the sample collector to the receiving section, an air-tight seal of the internal chamber of the dropper may be created. The air-tight seal can provide an air lock within the internal chamber of the dropper, retaining fluid sample within the internal chamber, preventing it leaking from the aperture of the dropper, even after the sealing of the aperture is released. The retention of the fluid sample can also be partly due to weak hydrostatic forces acting at the air-liquid interface at the aperture.

A user may overcome the retention forces that hold the fluid sample within the internal chamber of the dropper, and therefore dispense the fluid sample from the aperture of the dropper, by the application of the pressure to the flexible side walls of the dropper. Fluid sample may be expelled drop-wise from the aperture in a controlled manner, for example.

In one aspect, the present disclosure provides a sampling device for collecting and dispensing a sample, the sampling device comprising:

a sample collector comprising:
a cap; and
an insertion portion having a proximal end and a distal end, a proximal end of the insertion portion being connected to the cap and an absorbent portion being located at or adjacent the distal end of the insertion portion, the absorbent portion being configured to collect a sample;

a receiving section having an internal chamber, the sample collector being configured to be secured to the receiving section after collection of the sample, wherein the cap at least partially closes the internal chamber and the absorbent portion of the sample collector is received in the internal chamber;

wherein, when the absorbent portion is received in the internal chamber, the absorbent portion interferes with an extraction surface of the receiving section to cause extraction of sample from the absorbent portion into the internal chamber.

The interference between the absorbent portion and the extraction surface can assist with extraction (expression) of sample from the absorbent portion into the internal chamber. The extraction surface may be a surface of a wall that at least partially defines the internal chamber of the receiving section. The extraction surface may be provided at least in part by a bottom surface of the internal chamber. The extraction surface may be provided at least in part by side surfaces of the internal chamber.

The absorbent portion may deform when it interferes with the extraction surface. The length of the insertion portion may be such that, when the absorbent portion is received in the internal chamber, the absorbent portion cannot be accommodated in the internal chamber without deforming. The deformation may comprise bending, e.g. at an acute angle. For example, the absorbent portion may bend by an angle of at least 90 degrees, at least 120 degree or at least 150 degrees, although lower or higher angles may be employed. The bending may cause spring biasing of the absorbent portion against the extraction surface, causing a pressure to be applied between the absorbent portion and the extraction surface. The absorbent portion may deform by bending back and pressing against itself, which may further assist in extraction of the sample.

The absorbent portion may be elongate and the absorbent portion may bend at a central portion thereof. At least half of a length of the absorbent portion may be configured to interfere with the extraction surface. In general, a greater amount of extraction of the sample from the absorbent portion may be achieved by providing for a relatively large contact area and/or relatively high pressure between the absorbent portion and the extraction surface.

The absorbent portion may comprise absorbent material, such as cotton or another cellulosic or fiber-based absorbent material, surrounding a flexible rod portion. The absorbent portion may be configured as part of a swab, which may be supported by an elongate body, e.g. in a manner described above.

The sample collector may be configured to be secured to the receiving section after collection of the sample to form a dropper from which sample, e.g. fluid sample, is dispensable. The receiving section may include flexible side walls. The flexible side walls may be engageable by a user of the device, e.g. by a user's thumb and forefinger, to press the flexible side walls to cause dispensing of the sample from the internal chamber.

The cap may comprise at least one aperture through which fluid sample can be dispensed from the sampling device. The cap may comprise a lid that is movable between an open and closed position to selectively seal the aperture. A fluid path may be provided through the insertion portion at a proximal region of the insertion portion. This may ensure that the insertion portion does not obstruct flow of fluid sample from the internal chamber to the aperture. The proximal region of the insertion portion may comprise one or more openings adjacent the aperture. The insertion portion may comprise spaced apart legs that connect the insertion portion to the cap at connection positions that are spaced around the aperture.

The receiving section may comprise a liquid such as a buffer solution or other type of diluent in its internal chamber, e.g. prior to securing of the sample collector to the receiving section. The liquid may be deposited in the internal chamber during manufacture of the sampling device. A releasable seal may be located over an opening of the sealing section to seal the liquid in the internal chamber after manufacture. Alternatively, a user may deposit the liquid into the internal chamber prior to receipt of the sample collector. The user may deposit the liquid from a container such as a vial or ampoule. The container may be packaged with the sampling device. Additionally or alternatively, reagents may be located in the internal chamber. For example, reagents may be coated on or in a surface of the internal chamber or other item located in the internal chamber, e.g. prior to securing of the sample collector to the receiving section. Alternatively, a user may deposit the reagents into the internal chamber. The reagents may comprise particles, e.g. nanoparticles, fluorescent particles, quantum dots, etc., that are configured to label a target analyte in the sample for the purpose of performing a subsequent lateral flow test, for example. The reagents may be presented in a liquid, powder, gel, freeze-dried form or otherwise.

When the sample collector is secured to the receiving section, sample located on the absorbent portion of the sample collector may combine or react with the liquid and/or reagents to provide a more fluid version of the sample (described herein as a “fluid sample”) and/or a treated version of the sample.

In any aspect described herein, the sampling device may comprise a desiccant. The desiccant may be contained in a desiccant housing. The receiving section may comprise the desiccant housing, e.g. in a base thereof and/or underneath the internal chamber.

Following from this, in one aspect, the present disclosure provides a sampling device, the sampling device comprising:

a sample collector to collect a sample;

a receiving section, the sample collector being configured to be coupled to the receiving section after collection of the sample, the receiving section comprising a desiccant housing containing a desiccant.

The desiccant may maintain dryness of components of the receiving section and/or entire sampling device prior to use, including any reagents that may be located in the internal chamber prior to use, for example. Additionally or alternatively, the desiccant may maintain dryness of an environment surrounding the sampling device such as the inside of a packaging that may surround the sampling device prior to use. One or more open windows may be provided in the desiccant housing to ensure that condensation, water vapours or otherwise can reach the desiccant. The desiccant housing may be a cage or have a cage-like construction for housing the desiccant.

The securing between the sample collector and the receiving section may in accordance with aspects described above. For example, the sample collector may be secured to the receiving section by a screw-fitting. Rotation of the sample collector during screwing may further assist with extraction of the sample from the absorbent portion, as the absorbent portion rotates relative to the extraction surface.

In any aspects described herein, a length adapter may be provided that is configured to be coupled to the sample collector to adjust a length of the insertion portion that is insertable into a body cavity. The sample collector may adjust a maximum insertion depth of the absorbent portion into a cavity of a body. This may provide a safety mechanism and may enable the sample collector to be adapted for use in different body cavities and/or with differently sized subjects, preventing over-insertion of the sample collector into the body cavity. For example, the length adapter may be employed when the sample collector is to be used to collect a sample from an infant or child subject. The length adapter may be used with sample collectors as described above or with other sample collectors, which are not necessarily configured to be secured to a receiving section, for example.

Related to this, in one aspect of the present disclosure there is provided sample collector apparatus comprising:

a sample collector comprising:
a handle; and
an insertion portion having a proximal end and a distal end, a proximal end of the insertion portion being connected to the handle and an absorbent portion being located at or adjacent the distal end of the insertion portion;
wherein the sample collector is configured to collect a sample by inserting a length of the insertion portion including the absorbent portion into a body cavity; the apparatus further comprising:

a length adapter configured to be coupled to the sample collector to adjust a maximum insertion depth of the absorbent portion into the cavity.

In one aspect of the present disclosure there is provided a length adapter for use with a sample collector, the sample collector comprising:
a handle; and
an insertion portion having a proximal end and a distal end, a proximal end of the insertion portion being connected to the handle and an absorbent portion being located at or adjacent the distal end of the insertion portion;
wherein the sample collector is configured to collect a sample by inserting a length of the insertion portion including the absorbent portion into a body cavity;

the length adapter being configured to be coupled to the sample collector to adjust a maximum insertion depth of the absorbent portion into the cavity.

In any aspect disclosed herein, the length adapter may be configured to increase an effective diameter of a region of the insertion portion. The length adapter may be configured to be releasably coupled to the sample collector. The length adapter may be configured to be releasably coupled to a proximal region of the insertion portion. The length adapter may be configured to be coupled to the sample collector via an interference or friction fit. Additionally or alternatively, the length adapter may be configured to be coupled to the sample collector via a clip mechanism, a screw-fitting, a bayonet-fitting or otherwise.

The length adapter may be substantially annular. The length adapter may have inner side walls and outer side walls, the inner side walls defining a central, e.g. circular, opening through which the insertion portion is extended when the length adapter is coupled to the sample collector. The length adapter may have a split in the inner and outer side walls. The split may enable the walls of the length adapter to flex and therefore the central opening to increase in size, as a friction or interference fit is achieved with the sample collector. This may further provide for a spring-biased coupling between the length adapter and the sample collector.

The central opening may have an outer diameter as defined by the inner side walls that substantially corresponds to the diameter of an outer surface of part of the insertion portion. The outer side walls may have a diameter that substantially corresponds to the diameter of an outer surface of the handle. The outer side walls may have a diameter that is greater than a diameter of an opening to the body cavity, ensuring that the length adapter cannot enter the body cavity.

In accordance with preceding aspects, the handle may be provided by a cap. The sample collector may be configured to be coupled to a receiving section comprising an internal chamber, wherein the cap at least partially closes the internal chamber and the absorbent portion of the sample collector is received in the internal chamber.

In any aspects described herein, the sample may be dispensed from the sampling device on to a test device. The test device may be any type of test device configured to receive and test a sample, such as a lateral flow test device or otherwise.

The sample may be collected directly from a subject using the sample collector or may be collected, using the sample collector, from matter that has already been retrieved from a subject.

Reference herein to a “sample” should be understood as a reference to any sample derived from an animal or an environmental source. The sample may be a biological sample. The biological sample may be, for example, any material, biological fluid, tissue, or cell obtained or otherwise derived from a subject including, but not limited to, blood (including whole blood, leukocytes, peripheral blood mononuclear cells, plasma, or serum), sputum, mucus, nasal aspirate, urine, semen, saliva, meningeal fluid, lymph fluid, milk, bronchial aspirate, a cellular extract, brain tissue, or cerebrospinal fluid. The sample may include experimentally separated fractions of any of the preceding. For example, a blood sample can be fractionated into serum or into fractions containing particular types of blood cells, such as red blood cells or white blood cells (leukocytes). If desired, a sample may be a combination of samples from an individual, such as a combination of a tissue and fluid sample. A biological sample may also include materials containing homogenized solid material, such as from a stool sample, a tissue sample, or a tissue biopsy; or materials derived from a tissue culture or a cell culture. Thus, the term “sample” includes extracts and/or derivatives and/or fractions of the sample.

Moreover, as used herein, the term “subject” shall be taken to mean any animal including humans, for example a mammal. Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). For example, the mammal may be a human or primate. In a particular example, the mammal is a human.

In aspects of the present disclosure, use of an absorbent portion of a sample collector is described. It should be understood, however, that the sample collector according to any one or more aspects of the present disclosure may be configured to collect a sample without having an absorbent portion. A collection portion may be provide in place of the absorbent portion, for example, which is configured to receive a sample, e.g. by contacting the sample, but is not necessarily absorbent. The collection portion may be in the form of a scoop, spatula, tube, blade or otherwise.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments of the present disclosure are now described with reference to the accompanying Figures in which:

FIG. 1a shows a side view of a sampling device according to an embodiment of the present disclosure, FIG. 1b shows a cross-sectional view of the sampling device along line A-A of FIG. 1a, and FIG. 1c shows an expanded view of a portion of the sampling device of FIG. 1a;

FIG. 2 shows an exploded oblique view of the sampling device of FIG. 1a;

FIGS. 3a to 3e illustrate steps carried out in a method, according to an embodiment of the present disclosure, that uses the sampling device of FIG. 1a;

FIG. 4a shows a side view of a sampling device according to another embodiment of the present disclosure, and FIG. 4b shows a cross-sectional view of the sampling device of FIG. 4a;

FIG. 5a shows a side view of the sampling device of FIG. 4a in a further configuration and FIG. 5b shows a cross-sectional view of the sampling device of FIG. 5a;

FIG. 6 shows a cross-sectional side view of the sampling device of FIG. 4a in a further configuration;

FIG. 7a shows a side view of a sampling device, having a sample collector and receiving section in a separated configuration, according to an embodiment of the present disclosure and FIG. 7b shows a cross-sectional view of the sampling device along line C-C of FIG. 7a;

FIG. 8a shows a side view of the sampling device of FIG. 7a with the sample collector and the receiving section in a coupled configuration, and FIG. 8b shows a cross-sectional view of the sampling device along line A-A of FIG. 8a;

FIG. 9a. shows an alternative side view of the sampling device of FIG. 7a with the sample collector and the receiving section in a coupled configuration and with a lid of the sample collector open, and FIG. 9b shows a cross-sectional view of the sampling device of FIG. 9a;

FIG. 10 shows an exploded oblique view of the sampling device of FIG. 7a;

FIGS. 11a to 11e illustrate steps carried out in a method, according to an embodiment of the present disclosure, that uses the sampling device of FIG. 7a;

FIG. 12a shows a side view of sample collector apparatus according to an embodiment of the present disclosure, and FIG. 12b shows a cross-sectional view of the sample collector apparatus along line B-B of FIG. 12a;

FIG. 13 shows an oblique view of the sample collector apparatus of FIG. 12a with a sample collector and a length adapter of the apparatus in a separated configuration;

FIG. 14 illustrates a step carried out in a method, according to an embodiment of the present disclosure, that uses the sample collector apparatus of FIG. 12a; and

FIGS. 14a to 14c illustrate sample collector apparatuses according to embodiments of the present disclosure that have length adapters of different sizes.

### DESCRIPTION OF EMBODIMENTS

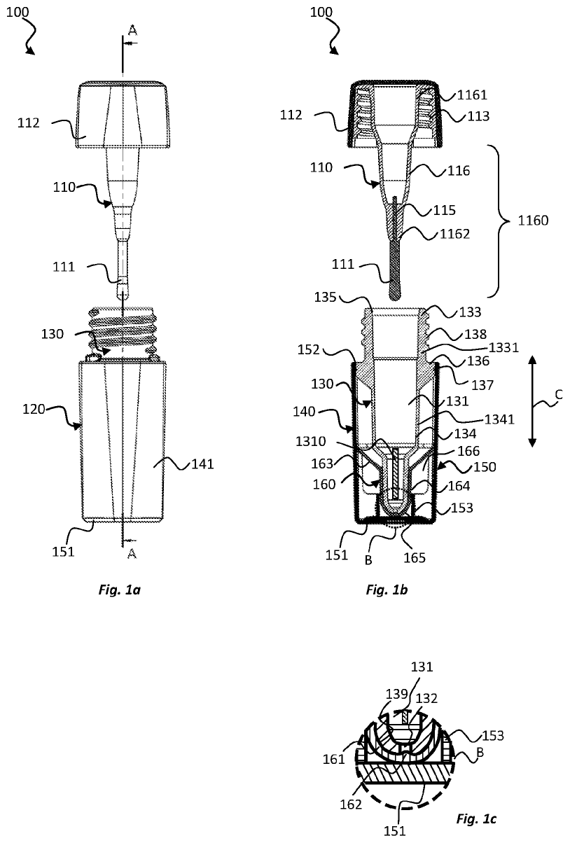

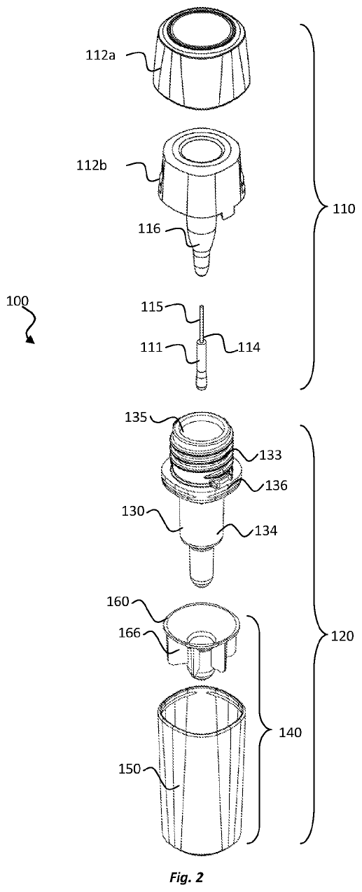

A sampling device according to an embodiment of the present disclosure is now described with reference to FIGS. 1a to 2. The sampling device 100 is for collecting a sample and dispensing a portion of the sample to a test device. Initially, the sampling device 100 is presented as two separate sections: a sample collector 110 and a receiving section 120.

The sample collector 110 has an absorbent portion 111 that is configured to receive a sample, such as a liquid sample, e.g., by rotating, wiping or pressing the absorbent portion against a part of the body from which the sample is to be received. The absorbent portion 111 is located at a first (distal) end of the sample collector 110, the sample collector also including a cap 112 at a second (proximal) end of the sample collector 110 that is opposite to the first end. The cap 112 is constructed in two parts including an outer part 112a and an inner part 112b. The absorbent portion 111 forms part of a swab 114, the swab 114 also including a rod 115 that is inserted into an opening at a distal end 1162 of an elongate body 116 that projects from a bottom surface of the inner cap 112b. The elongate body 116 is centrally located on the cap 112 and tapers from a proximal end 1161 connected to the cap 112 towards its distal end 1162. In combination, the elongate body 116 and the absorbent portion 111 can be considered to provide an insertion portion 1160 of the sample collector 110, a length of which insertion portion 1160 may be inserted into a body cavity such as a nasal cavity.

The receiving section 120 is configured to receive the sample collector 110 after the collection of a sample. As illustrated in FIG. 1b, the receiving section 120 includes a dropper 130 and a base 140, the dropper initially being releasably engaged with the base 140. The receiving by the receiving section 120 of the sample collector 110 involves securing the sample collector 110 to the dropper 130 and specifically screwing the sample collector 110 to the dropper 130 in this embodiment.

The dropper 130 defines an internal chamber 131 in which the absorbent portion 111 of the sample collector 110 is received, via a main opening 135 of the dropper 130, when the sample collector 110 is secured to the dropper 130. As best seen in FIG. 1c, which shows an enlargement at region B of FIG. 1b, the dropper 130 has an aperture 132 to dispense the sample from its internal chamber 131.

When the sample collector 110 is secured to the dropper 130, the main opening 135 of the dropper 130 is closed or sealed by the sample collector 110. The closing or sealing is achieved by a combination of the elongate body 116 extending through the opening 135 of the dropper 130 and contacting inner side walls adjacent the opening 135 in a tight fit manner, and also by a screw-fitting 113, 138 between the cap 112 and the dropper 130.

The base 140 includes a cup 150, the cup 150 having a substantially flat bottom wall 151 for supporting the base 140 on a surface such as a table or desk top. The cup 150 houses at least part of the dropper 130, in order to protect the dropper 130 and prevent dispensing of sample from the dropper 130 during initial use of the sampling device 100.

To prevent dispensing of sample from the dropper 130, at least during initial use of the sampling device 100, the base 140 is configured to seal the aperture 132 of the dropper 130 when the dropper 130 is releasably engaged to the base 140. With reference to FIG. 1c, to effect the sealing, the base 140 has a contoured sealing surface 161 at a lower end thereof. Adjacent the aperture 132, the dropper 130 has a contoured outer surface 139 and the contoured sealing surface 161 is configured to contact and conform to the contoured outer surface 139. The sealing surface 161 also includes a projection 162 that covers and/or projects into the aperture 132 of the dropper 130.

While sealing of the aperture is achieved using a sealing surface of the base in this embodiment, in alternative embodiments sealing may be achieved by other means. For example, a sealing surface may be provided by a plug that is at least partially inserted into the aperture to seal the aperture, which plug may remain in place upon release of the dropper from the base and may be removed, e.g. manually, when releasing of the seal is desired. The plug may be attached to the dropper, e.g. via a hinge or a frangible element.

In the present embodiment, the base 140 includes two separate components, the cup 150 as described above and an insert 160 that is located inside the cup 150 (see also FIG. 2). In this embodiment, the insert 160 comprises the sealing surface 161. The insert 160 has a conical upper section 163, a cylindrical middle section 164, and a dome-shaped lower section 165. The middle and lower sections 164, 165 provide the sealing surface 161. The conical upper section 163 helps guide the end of the dropper into the middle and lower sections 164, 165 during assembly of the sampling device 100. For structural stability, and to prevent movement of the insert 160 inside the cup, a plurality of radial supports 166 are provided on an outer surface of the middle and upper sections 163, 164 of the insert 160. Moreover, a hollow cylindrical section 153 is provided on an internal surface of the bottom wall 151 of the cup 150 that is sized to receive the lower section 165 of the insert 160 in a tight-fit manner.

In this embodiment, the dropper 130 has an upper section 133 and a lower section 134. The upper section 133 of the dropper 130 includes the main opening 135 through which the absorbent element 111 of the sample collector 110 can enter the internal chamber 131 of the dropper 130. The lower section 134 of the dropper 130 has the aperture 132 to dispense the sample from the dropper 130. The lower section 134 is relatively flexible in comparison to the upper section 133, e.g., as a result of being formed of thinner walls 1341 than walls 1331 that form the upper section 133. This flexibility can enable the walls 1341 of the lower section 134 to be pressed together by hand, e.g. to force sample through the aperture 132 of the dropper during use of the sampling device.

Once the sealing of the aperture 132 of the dropper 130 is released, fluid may initially be prevented from leaking through the aperture 132 by the creation of an air-tight seal of the internal chamber 131 of the dropper 130 when the sample collector 110 is secured to the dropper 130. The air-tight seal can provide an air lock within the internal chamber 131 retaining fluid sample within the internal chamber 131. The retention of the fluid can also be partly due to weak hydrostatic forces acting at the air-liquid interface at the aperture 132. The pressing of the walls 1341 by the user can overcome the retention forces that hold the fluid sample within the internal chamber 132, enabling dispensing of the sample. Fluid sample can be expelled drop-wise from the aperture 132 in a controlled manner, for example.

The dropper 130 also includes a collar 136 that is located between the upper section 133 and the lower section 134. The collar 136 extends radially outwardly of the upper and lower sections 133, 134 and contacts an inner surface of the base 140 (specifically an inner surface of the cup 150 in this embodiment) to releasably engage the dropper 130 with the base 140. The collar 136 contacts the inner surface of the cup 150 adjacent an upper opening 152 of the cup 150. The releasable engagement between the collar 136 and the cup 150 is achieved via a snap-fitting 137. The snap-fitting 137 includes a detent and a projection that releasably locates in the detent. In this embodiment, the detent is located on the collar 136 and the projection is located on the inner surface of the cup 150, although the locations of the detent and projection can be exchanged. The releasable engagement between the dropper 130 and the base 140 can be overcome by applying opposing axial forces to the dropper and the base (i.e. by pulling the dropper and the base apart in the axial direction of the sampling device, generally as indicated by arrow C in FIG. 1b).

As indicated above, the sample collector 110 and the dropper 130 are secured to each other by a screw-fitting. In particular, in this embodiment, the upper section 133 of the dropper 130 comprises a screw thread 138 for screwing to a complimentary screw thread 113 of the sample collector 110. The complimentary screw thread 113 is located on an inner surface of the cap 112. The screw-fitting between the sample collector 110 and the dropper 130 provides for a secure connection between the sample collector 110 and the dropper 130, particularly in the axial direction C of the sampling device 100. In general, after securing together, it is not possible to pull the sample collector 110 and the dropper 130 apart by applying opposing axial forces to the sample collector 110 and the dropper 130.

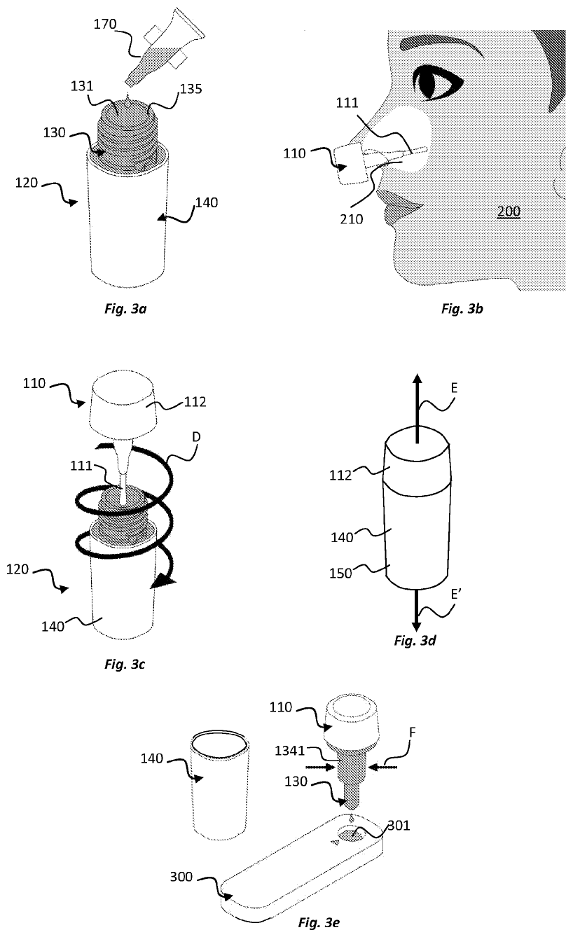

A method of use of the sampling device 100 is now described with reference to FIGS. 3a to 3e. With reference to FIG. 3a, a liquid such as a buffer solution is deposited into the receiving section 120 of the sampling device 100 and specifically so that the fluid partially fills the internal chamber 131 of the dropper 130 of the receiving section 120 while the dropper 130 is engaged with the base 140. The liquid may be deposited in the internal chamber 131 during manufacture of the sampling device 100, with a releasable sealing layer being located, for example, over the main opening 135 of the dropper to prevent spilling of the fluid prior to use. Alternatively, a user may deposit the fluid into the internal chamber 131 immediately prior to use, e.g. by releasing the fluid from a vial or ampoule 170 that may be packaged with the sampling device 100 or otherwise. Additionally or alternatively, reagents may be located in the internal chamber 131. For example, reagents may be coated on or in a surface of the internal chamber 131 or other item located in the internal chamber 131, e.g. prior to securing of the sample collector 110 to the dropper 130. The reagents may comprise particles that are configured to label a target analyte in the sample for the purpose of performing a subsequent lateral flow test, for example. The reagents may be in a liquid, powder, gel, freeze-dried form or otherwise.

With reference to FIG. 3b, prior to or after deposition of the liquid in the receiving section 120, the sample collector 110 is used to receive a sample from a subject 200. In the illustrated example, an intra-nasal sample is received by extending the absorbent portion 111 of the sample collector 110 into the nasal cavity 210 of the subject 200. A user can hold the cap 112 of the sample collector 110 when extending the absorbent portion 111 into the nasal cavity 210 and, in this regard, the cap 112 acts as a handle for the sample collector 110.

With reference to FIG. 3c, after receipt of sample on the absorbent portion 111 of the sample collector 110, with liquid located in the internal chamber of the dropper 130 and while the dropper remains engaged with the base 140, the absorbent portion 111 is extended into the internal chamber 131 of the dropper 130. During this process, the sample collector 110 is secured to the dropper 130 by screwing the sample collector to the dropper 130. The screwing action, indicated by Arrow D in FIG. 3c, causes rotation of the absorbent portion 111 within the internal chamber 131. The rotation assists with extraction of the sample from the absorbent portion 111 and mixing of the sample with the liquid contained in the dropper 130, forming a fluid sample mixture in the internal chamber 131 of the dropper 130. As the absorbent portion 111 rotates, it is moved against a protrusion, and particularly a rib 1310, that is positioned on an inner surface of a wall of the dropper 130 (see also FIG. 1b). Frictional and/or shear forces between the absorbent portion and the rib 1310 can assist with the extraction of the sample.

The sample collector 110, dropper 130 and base 140 may remain secured/engaged together, as illustrated in FIG. 3d, for a period of time sufficient to form a desired fluid sample mixture, e.g. through an incubation processes or otherwise. Once the fluid sample has been formed, the dropper 130, with fluid sample therein can be released from the base 140 by gripping the cap 112 of the sampling collector 110 secured to the dropper 130, and gripping the cup 150 of the base 140, and pulling the cap 112 and the cup 150 in opposite directions, general as indicated by arrows E and E′ in FIG. 3d.

The release of the dropper 130 from the base causes unsealing of the aperture 132 of the dropper 130 and exposes the sidewalls 1341 of the dropper 130. As illustrated in FIG. 3e, a user can apply compression, generally as illustrated by arrows F, to the sidewalls 1341 of the dropper 130, forcing fluid sample to be dispensed through the aperture 132. The fluid sample may be deposited in a controlled manner at a desired location, e.g. on a receiving portion 301 of a test device 300 as illustrated in FIG. 3e. The test device may be a lateral flow test device or otherwise.

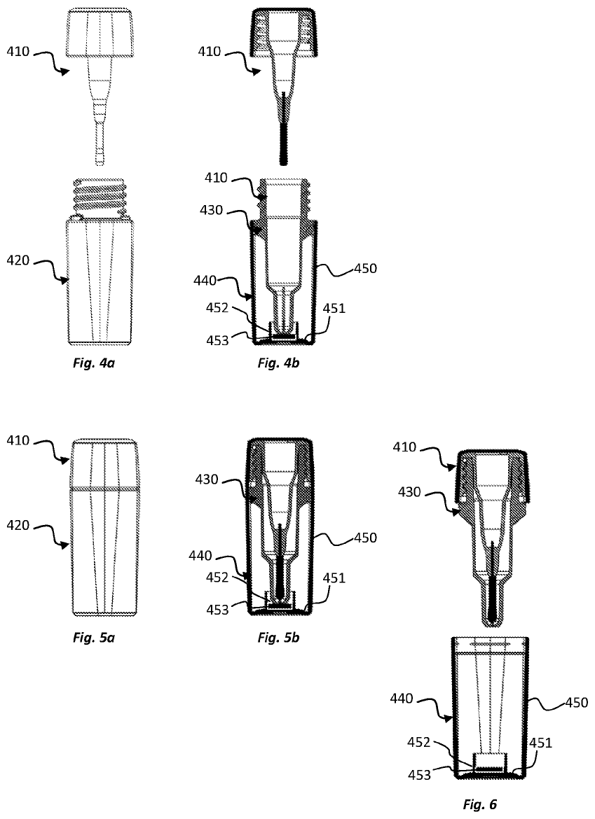

A sampling device 400 according to an alternative embodiment of the present disclosure is illustrated in FIGS. 4a to 6. The configuration and function of the sampling device 400 is very similar to the sampling device 100 described above with reference to FIGS. 1a to 3e and again includes a sample collector 410 and a receiving section 420, the receiving section including a dropper 430 and a base 440. FIGS. 4a to 6 provide views of the sampling device 400 prior to the securing of the sample collector 410 to the dropper (FIGS. 4a and 4b), after securing of the sample collector 410 to the dropper 430 (FIGS. 5a and 5b) and after release of the dropper 430 from the base 440 while the dropper 430 is secured to the sample collector 410 (FIG. 6).

The difference between the sampling device 400 of the present embodiment and the sampling device 100 of the preceding embodiment is primarily in relation to the base 440, which does not include the same insert for sealing the aperture of the dropper 430. Rather, on an inner bottom surface 451 of a cup 450 that forms the base 440, a receptacle 452 is provided in which a gasket 453 is located. The gasket 453 is a foam disk in this embodiment. The gasket 453 is configured to seal the aperture of the dropper 430, preventing any leakage of fluid therefrom until an airlock is formed by the action of securing the sample collector 410 to the dropper 430.

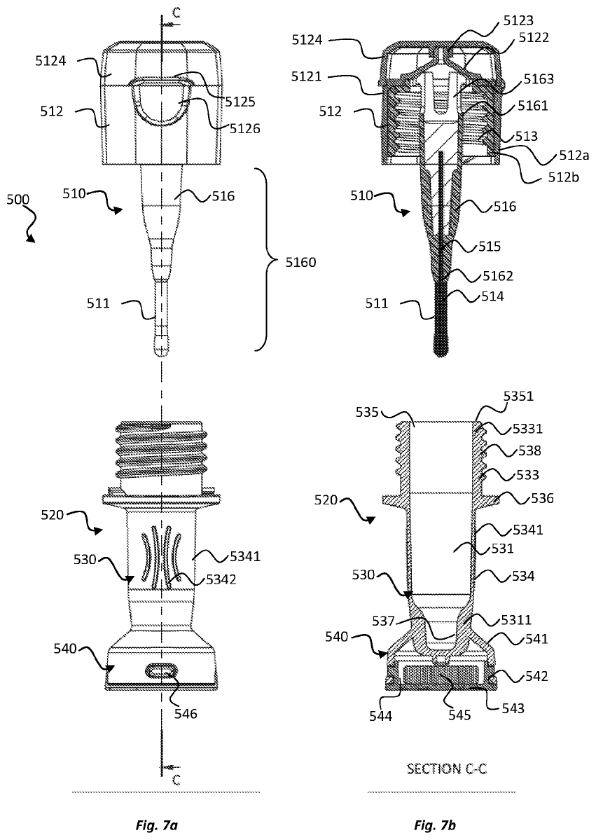

A sampling device 500 according to another embodiment of the present disclosure is now described with reference to FIGS. 7a to 10. The sampling device 500 is again for collecting a sample and dispensing a portion of the sample to a test device. Initially, the sampling device 500 is presented as two separate sections as shown in FIGS. 7a and 7b, a sample collector 510 and a receiving section 520.

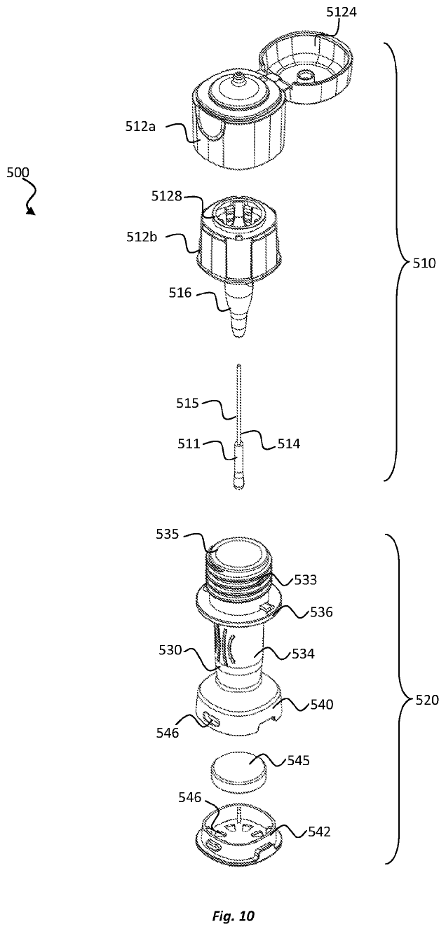

The sample collector 510 has an absorbent portion 511 that is configured to receive a sample, such as a liquid sample, e.g., by rotating, wiping or pressing the absorbent portion 511 against a part of the body, e.g. within a cavity such as a nasal cavity, from which the sample is to be received. The absorbent portion 511 is located at a first (distal) end of the sample collector 510, and a cap 512 is located at a second (proximal) end of the sample collector 510 that is opposite the first end. As best seen in FIG. 10, the cap 512 is constructed in two parts including an outer part 512a and an inner part 512b. The absorbent portion 511 forms part of a swab 514, the swab 514 also including a rod 515 that is inserted into an opening at a distal end 5162 of an elongate body 516 that projects from an inner surface of the inner cap 512b. The elongate body 516 is centrally located on the cap 112 and tapers from a proximal end 5161 connected to the cap 512 towards its distal end 5162. In combination, the elongate body 516 and the absorbent portion 511 can be considered to provide an insertion portion 5160 of the sample collector 510, a length of which insertion portion 5160 may be inserted into a body cavity such as a nasal cavity.

The receiving section 520 is configured to receive the sample collector 510 after the collection of a sample. The receiving section 520 includes a dropper body portion 530 and a base 540. The receiving by the receiving section 520 of the sample collector 510 involves securing (coupling) the sample collector 510 to the dropper body portion 530 and specifically screwing the sample collector 510 to the dropper body portion 530 in this embodiment, although other securing techniques may be employed. A screwing mechanism is provided by complimentary screw threads 513, 538 disposed on the cap 512 and the dropper body portion 530. A seal is provided between the cap 512 and the dropper body portion 530 by a first sealing engagement between a rim 5351 surrounding the opening 535 of the dropper portion 530 and a distal facing inner surface 5127 of the inner cap 512b and by a second sealing engagement between a proximally projecting rim 5128 of the inner cap 512b and an inner shoulder 5129 of the outer cap 512b that surrounds and/or partially defines the nozzle 5122.

When secured together, the sample collector 510 and the receiving section 520 form a dropper. In this embodiment, an aperture 5123 of the dropper, through which sample can be dispensed, is provided in the cap 512 rather than in part of the receiving section 520, as is discussed in more detail below. The seal provided between the cap 512 and the dropper body portion 530 ensures that sample can exit the dropper via the aperture 5123 only.

The dropper body portion 530 defines an internal chamber 531 in which the absorbent portion 511 of the sample collector 510 is received, via a main opening 535 of the body portion 530, when the sample collector 510 is secured to the body portion 530. When the sample collector 510 is secured to the dropper body portion 530, the main opening 535 of the body portion 530 is closed or sealed by the sample collector 510 and in particular by the cap 512 of the sample collector 510.

The base 540 has a cup shape, formed by a first portion 541 that extends from and is integrally formed with the dropper body portion 530 and a second portion 542 that couples to the first portion 541 and which has a substantially flat bottom wall or bottom edge 543 for supporting the base 540, and therefore the entire receiving section 520, on a surface such as a table or desk top. In this embodiment, the base 540 provides a desiccant housing 544 for receipt of a desiccant 545. The desiccant 545 may maintain dryness of components of the receiving section 520 and/or entire sampling device 500 prior to use, including any reagents that may be located in the internal chamber 531 prior to use, for example. Additionally or alternatively, the desiccant may maintain dryness of an environment surrounding the sampling device 500 such as the inside of a packaging that may surround the sampling device 500 prior to use. The base or desiccant housing 540 includes one or more windows 546 to ensure that condensation, water vapours or otherwise can reach the desiccant 545. The windows may be located in the first and/or second portion 541, 542 of the base.

In this embodiment, the dropper body portion 530 of the receiving section 520 has an upper section 533 and a lower section 534. The upper section 533 of the dropper body portion 530 includes the main opening 535 through which the absorbent element 511 of the sample collector 510 can enter the internal chamber 531 of the dropper body portion 530. The lower section 534 defines at least a portion of the internal chamber 531 in which the absorbent portion 511 is received. The lower section 534 is relatively flexible in comparison to the upper section 533, e.g., as a result of being formed of thinner walls 5341 than walls 5331 that form the upper section 533. This flexibility can enable the walls 5341 of the lower section to be pressed together by hand, e.g. to force sample through the aperture 5123 during use of the sampling device 500. Finger grips and/or markings 5342 may be provided on an outer surface of the walls 5341 to indicate where a user may press the walls 5341.

The cap 512 includes a main body 5121 that has a nozzle 5122 at a top end, the nozzle defining the aperture 5123 through which sample can be dispensed from the dropper that is formed by the sample collector 510 and the receiving section 520 when they are secured together. The nozzle 5122 is centrally located at the top end of the cap 512. The cap 512 includes a lid 5124 that is movable between an open and closed position, at the top end of the cap 512, to selectively seal the aperture 5123. The lid 5124 in this embodiment is hingedly connected to the body 5121 of the cap 512. A user can engage a lip 5125 of the lid 5124 that is accessible adjacent a shallow recess 5126 in the body 5121.

When the aperture 5123 of the sampling device 500 is released by opening the lid 5124, an air lock may retain fluid sample, even when the device 500 is inverted from the position shown in FIGS. 7a to 9b. The retention of the fluid can also be partly due to weak hydrostatic forces acting at the air-liquid interface at the aperture 5123. The pressing of the walls 5341 by the user can overcome the retention forces that hold the fluid sample, enabling dispensing of the sample, which may be a fluid sample. Fluid sample can be expelled drop-wise from the aperture 5123 in a controlled manner, for example.

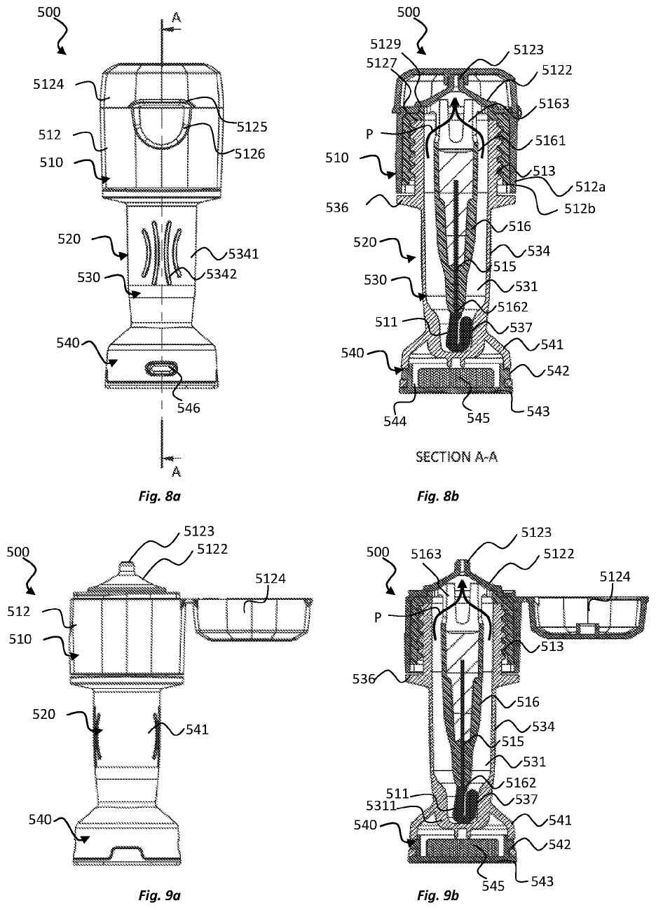

So that the insertion portion 5160, and specifically the elongate body 516 of the insertion portion 5160, does not obstruct flow of fluid from the internal chamber 531 to the aperture 5123, a fluid path is provided through the elongate body 516 at a proximal region of the elongate body 516 positioned adjacent the aperture 5123, the fluid path being generally as indicated by arrows P in FIGS. 8b and 9b. The fluid path P is provided in this embodiment by providing an at least partially hollow elongate body 516 that has openings in its side walls adjacent the aperture 5123. The openings are provided in this embodiment between spaced apart legs 5163 of the elongate body 516, which legs connect the elongate body 516 to the cap 512 at connection positions that are spaced around the aperture 5123.

As indicated above, the sample collector 510 and the receiving section 530 are secured to each other by a screw-fitting. In particular, in this embodiment, the upper section 533 of the dropper body portion 530 comprises a screw thread 538 for screwing to a complimentary screw thread 513 of the sample collector 510. The complimentary screw thread 513 is located on an inner surface of the cap 512. The screw-fitting between the sample collector 510 and the dropper body portion 530 provides for a secure connection between the sample collector 510 and the receiving section 520, particularly in the axial direction of the sampling device 500. In general, after securing together, it is not possible to pull the sample collector 510 and the receiving section 520 apart by applying opposing axial forces to the sample collector 510 and the receiving section 520.

The dropper body portion 530 includes a collar 536 that is located between the upper section 533 and the lower section 534. The collar 536 extends radially outwardly of the upper and lower sections 533, 534 and may provide an axial stop for the screwing of the cap 512 to the dropper body portion 530.

As seen in FIGS. 8a to 9b, when the absorbent portion 511 is received in the internal chamber 531, the absorbent portion 511 interferes with a wall of the receiving section 520 and specifically, at least in this embodiment, the absorbent portion 511 is pressed against an extraction (or expression) surface 537 of one or more walls defining the internal chamber 531. In this embodiment, the extraction surface 537 is provided at least in part by a bottom surface of the internal chamber 531 and at least in part by side surfaces of the internal chamber 531. The interference can assist with extraction (expression) of sample from the absorbent portion 511 into the internal chamber 531.

A greater amount of extraction of the sample from the absorbent portion 511 can be achieved by providing for a relatively large contact area and/or relatively high pressure between the absorbent portion 511 and the extraction surface 537. In the present embodiment, one or both of these features may be achieved by providing, in effect, an absorbent portion 511 that can be considered too long for the receiving chamber 531, or that can be considered to extend too far from the cap 512 to be received in its natural state within the receiving chamber 531, when the sample collector 510 and the receiving section 520 are secured to each other. When the sample collector 510 is secured to the receiving section 520, the absorbent portion 511 is therefore forced to deform, e.g. buckle and/fold, by pressing against the extraction surface 537, in order to fit within the internal chamber 531. The deformation can be such that the absorbent portion 511 bends by an angle of over 90 degrees, over 120 degrees, over 150 degrees or higher, e.g. at a central portion thereof. The absorbent portion 511 may be generally elongate and flexible and may include absorbent material surrounding a flexible rod portion, e.g. a flexible distal portion of the rod 515. The absorbent material may be cotton or another cellulosic or fiber-based absorbent material. The deformation may be such that the absorbent portion 511 bends back and presses against itself, in addition to the extraction surface 537, to further assist with extraction of sample from the absorbent portion 511. At least in the present embodiment, this degree of bending is achieved through the provision of a narrow section 5311 of the receiving chamber 531 at a lower end of the internal chamber 531. The screwing action between the sample collector 510 and the receiving section 520 can also assist with extraction of the sample as it causes the absorbent portion 511 to progressively engage the extraction surface 537 in an axial direction and to rotationally wipe against the extraction surface 537, increasing frictional and/or shear forces between the absorbent portion 511 and the extraction surface 537.

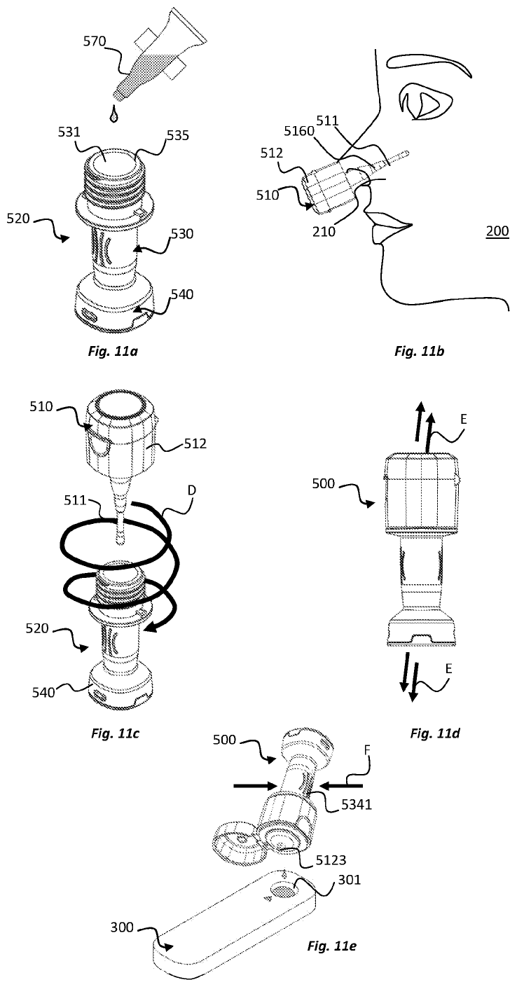

A method of use of the sampling device 500 is now described with reference to FIGS. 11a to 11e. With reference to FIG. 11a, a liquid such as a buffer solution and/or diluent is deposited into the receiving section 520 of the sampling device 500 and specifically so that the liquid partially fills the internal chamber 531 of the receiving section 520. The liquid may be deposited in the internal chamber 531 during manufacture of the sampling device 500, with a releasable sealing layer being located, for example, over the main opening 535 of the dropper to prevent spilling of the liquid prior to use. Alternatively, a user may deposit the liquid into the internal chamber 531 immediately prior to use, e.g. by releasing the liquid from a vial or ampoule 170 that may be packaged with the sampling device 500 or otherwise. Additionally or alternatively, reagents may be located in the internal chamber 531. For example, reagents may be coated on or in a surface of the internal chamber 531 or other item located in the internal chamber, e.g. prior to securing of the sample collector 510 to the receiving section 520. The reagents may comprise particles that are configured to label a target analyte in the sample for the purpose of performing a subsequent lateral flow test, for example. The reagents may be in a liquid, powder, gel, freeze-dried form or otherwise.

With reference to FIG. 11b, prior to or after deposition of the liquid in the receiving section 520, the sample collector 510 is used to receive a sample from a subject 200. In the illustrated example, an intra-nasal sample is received by extending the insertion portion 5160 including the absorbent portion 511 of the sample collector 510 into the nasal cavity 210 of the subject 200. A user can hold the cap 512 of the sample collector 510 when extending the absorbent portion 511 into the nasal cavity 210 and, in this regard, the cap 512 acts as a handle for the sample collector 510.

With reference to FIG. 11c, after receipt of sample on the absorbent portion 511 of the sample collector 510, with liquid located in the internal chamber 531 of the receiving section 520, the absorbent portion 511 is extended into the internal chamber 531. During this process, the sample collector 510 is secured to the receiving section 520 by screwing the sample collector 510 to the receiving section 520. The screwing action, indicated by Arrow D in FIG. 11c, causes rotation of the absorbent portion 511 within the internal chamber 531. The rotation assists with extraction of the sample from the absorbent portion 511 and mixing of the sample with the liquid contained in the internal chamber 531, forming a fluid sample mixture in the internal chamber 531. As the absorbent portion 511 rotates, it is advanced into the internal chamber 531, causing it to deform against and progressively engage the extraction surface 537 and to rotationally wipe against the extraction surface 537 as discussed above.

After securing the sample collector 510 to the receiving section 520, a dropper is effectively formed by the device 500, generally as illustrated in FIG. 11d. To further assist with mixing of the sample and the liquid contained in the internal chamber 531, the device 500 may be shaken as indicated by arrows E in FIG. 11d. Additionally, or alternatively, the sample collector 510 and receiving section 520 may maintained in the state shown in FIG. 11d for a period of time sufficient to form a desired fluid sample mixture, e.g. through incubation processes or otherwise.

Once the fluid sample has been formed, the device 500 can be inverted as shown in FIG. 11e. Before, during or after inversion, the lid 5124 of the cap can be opened by a user to unseal the aperture 5123. As illustrated in FIG. 11e, a user can apply compression, generally as illustrated by arrows F, to the sidewalls 5341 of the dropper body portion 530, forcing fluid sample to be dispensed through the aperture 5123. The fluid sample may be deposited in a controlled manner at a desired location, e.g. on a receiving portion 301 of a test device 300 as illustrated in FIG. 11e. The test device may be a lateral flow test device or otherwise.

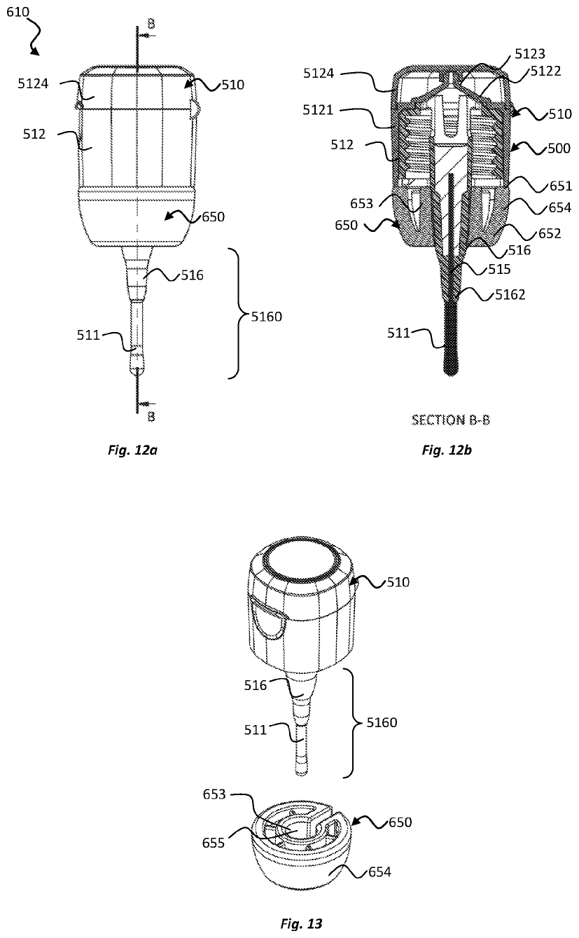

A sample collector apparatus 610 according to an embodiment of the present disclosure is now described with reference to FIGS. 12a to 13. The sample collector apparatus 610 of this embodiment comprises a sample collector, such as the sample collector 510 as described above with reference to FIGS. 7a to 10, in combination with a length adapter 650. The length adapter 650 is usable to limit the degree by which the insertion portion 5160 of the sample collector 510 may be inserted into a cavity such as nasal cavity of a subject. For example, it may provide for adjustment of a maximum insertion depth of the absorbent portion 511 into the cavity.

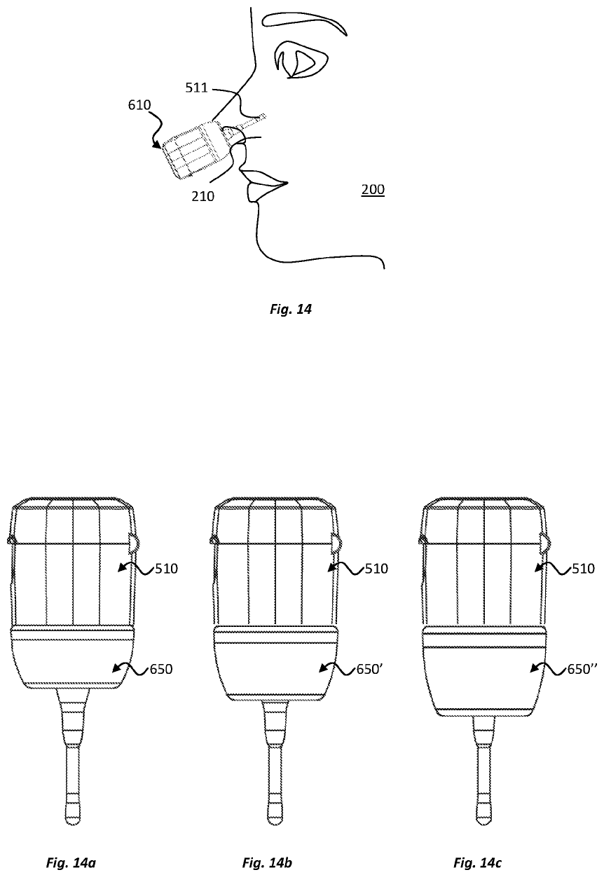

In general, the insertion portion 5160, including the absorbent portion 511 and at least part of the elongate body 516 that supports the absorbent portion 511, is configured to be inserted into a cavity such as a nasal cavity of a subject. On the other hand, the cap 512 may have a diameter that is too large to be inserted into the cavity. The length adapter 650 may have a diameter that is similar to the cap and/or may also be too large to insert into the cavity. The length adapter 650 may provide for an extension of the cap 512. The length adapter 650 may provide for an increase in the an effective diameter of a region of the insertion portion 5160 that is located proximally of the absorbent portion 511. The length adapter 650 is coupled to the sampler collector 510 and particularly so that it locates over a portion of the insertion portion 5160 and in this embodiment a portion of the elongate body 516 that supports the absorbent portion 511. In this regard, when used, the length adapter 650 may be considered to shorten the insertable length of the insertion portion 5160, reducing the degree by which it can be inserted in the cavity 210 of the subject 200 and therefore the maximum insertion depth of the absorbent portion 511 as shown in FIG. 14 (cf. FIG. 11b).

The length adapter 650 is arranged to locate between the cap 512 and the absorbent portion and to at least partially surround the elongate body 516. A proximal end surface 651 of the length adapter 650 may abut against the cap 512. The cap 512 and the length adapter 650 may each have a substantially circular circumference. The proximal end surface 651 of the length adapter 650 may have a diameter that is substantially the same as the diameter of the distal end of the cap 512 such that, when in abutment, there is a relatively smooth transition between outer walls of the cap 512 and the length adapter 650. A distal end surface or region 652 of the length adapter 650 has curved and/or smooth walls to reduce risk of trauma or discomfort to the user if pressed against the user's skin or other tissue. The distal end surface or region 652 of the length adapter may be considered an insertion limiting surface.

In this embodiment, the length adapter 650 is releasably coupled to the sample collector 510. In alternative embodiments, the length adapter 650 may be arranged to be permanently coupled to the sample collector 510.

The length adapter 650 in this embodiment is substantially annular and formed of an, e.g. hollow, body with inner side walls 653 and outer side walls 654. The inner walls 653 define a central, e.g. circular, opening 655 through which the elongate body 516 of the sample collector 510 is extended when the length adapter 650 is coupled to the sample collector 510. The central circular opening 655 may have an outer diameter as defined by the inner walls 653 that substantially corresponds to the diameter of an outer surface of part of the insertion portion 5160, such as part of the elongate body 516. For example, the inner walls 653 may be arranged to slide against and abut an outer surface of the elongate body 516, providing for an interference or friction-fit between the length adapter 650 and the elongate body 516. The length adapter 510 has a split in the side walls 653, 654 in this embodiment, enabling the walls 653, 654 to flex and therefore the central opening 655 to increase in size, as the friction fit is achieved. This may further provide for a spring-biased coupling between the length adapter 650 and the sample collector 510. Additionally or alternatively, a clip, bayonet-fitting or other securing mechanism may be provided for coupling the length adapter 650 to the sample collector 510.

With reference to FIGS. 14a to 14c, length adapters 650, 650′, 650″ having different sizes (e.g. depths, extending in the axial direction of the sample collector 510) may be provided to vary the degree by which the insertion portion, including the elongate body and/or absorbent portion can be inserted in the cavity of the subject.

In one embodiment a kit may be provided comprising a sample collector 510 in combination with one or more length adapters 650, 650′, 650″, optionally contained within a single package. Where more than one length adapter 650 is provided, each length adapter 650 may have a different size and may be selectively coupled to the sample collector 510, e.g. by a user or during manufacture, depending on the nature of the cavity into which the sample collector 510 is to be inserted and/or the size of the subject. One or more length adapters 650, 650′, 650″ may be provided that adapt the sample collector for use with an infant subject, a junior subject, a youth subject and/or an adult subject, respectively. However, when the sample collector is to be used with an adult subject, a length adapter may alternatively be excluded from use.

After a sample has been collected, the length adapter 650 may be decoupled from the sample collector 510, prior to the sample collector 510 being secured to a receiving section 520, e.g. in accordance with discussions above with reference to FIG. 11c. Alternatively, a receiving section may be provided that is configured to accommodate a length adapter when a sample collector apparatus is secured thereto. The length adapter 650 has been described above with reference to a sample collector 510 having a cap 512 that may be coupled to a receiving portion, e.g. to form a dropper. However, in alternative embodiments a sample collector may be provided in which the cap is replaced with a more general handle, which may have similar dimensions to the cap, but may not include a screw thread or other securing mechanism, e.g. if the sample collector is not required to be secured to or seal any portion of a receiving section.

In embodiments of the present disclosure, use of an absorbent portion of a sample collector is described. It should be understood, however, that the sample collector according to any one or more embodiments may be configured to collect a sample without having an absorbent portion. A collection portion may be provide in place of the absorbent portion, for example, which is configured to receive a sample, e.g. by contacting the sample, but is not necessarily absorbent. The collection portion may be in the form of a scoop, spatula, tube, blade or otherwise.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A sampling device for collecting and dispensing a sample, the sampling device comprising:
a sample collector having an absorbent portion configured to collect a sample;
a receiving section configured to receive the sample collector after the collection of a sample, the receiving section comprising:
a dropper, the sample collector configured to be secured to the dropper, the dropper defining an internal chamber in which the absorbent portion of the sample collector is received when the sample collector is secured to the dropper and having an aperture to dispense the sample from the internal chamber; and
a base releasably engaged with the dropper and at least partially housing the dropper, wherein the dropper is configured to be released from the base to allow dispensing of the sample from the dropper.

2. The sampling device of claim 1, wherein the dropper is configured to be released from the base while the sample collector and the dropper are secured together.

3. The sampling device of claim 1 or 2, wherein the dropper is configured to be released from the base by pulling the dropper and base apart in an axial direction of the sampling device.

4. The sampling device of claim 3, wherein a minimum axial force to release the dropper from the base is lower than a minimum axial force to release the securing of the dropper to the sample collector.

5. The sample device of any one of the preceding claims, wherein the dropper and the base are releasably engaged with each other by a snap-fitting.

6. The sampling device of any one of the preceding claims, wherein the sample collector and the dropper are secured to each other by a screw-fitting.

7. The sampling device of any one of the preceding claims, wherein the dropper comprises an upper section and a lower section, wherein the upper section has an opening for the sample collector to access the internal chamber of the dropper and the lower section has the aperture to dispense the sample from the dropper.

8. The sampling device of claim 7, wherein the dropper comprises a collar located between the upper section and the lower section, the collar extending radially outwardly of at least the lower section of the dropper, to contact an inner surface of the base to releasably engage the dropper with the base.

9. The sampling device of claim 7 or 8, wherein, when the dropper is releasably engaged with the base, the lower section of the dropper is located inside the base and the upper section of the dropper protrudes from the base.

10. The sampling device of claim 7, 8 or 9, wherein the upper section of the dropper comprises a screw thread for screwing to a complimentary screw thread of the sample collector.

11. The sampling device of any one of the preceding claims, wherein the sample collector comprises a cap configured to at least partially close the internal chamber of the dropper when the sample collector is secured to the dropper.

12. The sampling device of claim 10, wherein the sample collector comprises a cap configured to at least partially close the internal chamber of the dropper when the sample collector is secured to the dropper, the complimentary screw thread being located on a surface of the cap.

13. The sampling device of any one of the preceding claims, wherein the sample collector comprises an elongate body that projects from the cap and that is configured to at least partially close the internal chamber of the dropper when the sample collector is secured to the dropper.

14. The sampling device of any one of the preceding claims, wherein the sample collector comprises a swab, the swab comprising the absorbent portion.

15. The sampling device of claim 14, wherein the swab comprises a rod and the absorbent portion is located at an end of the rod.

16. The sampling device of any one of the preceding claims, wherein the base comprises a cup, the cup having a bottom surface to support the sampling device on a surface and a top opening for receiving the dropper.

17. The sampling device of any one of the preceding claims, wherein the base comprises a sealing surface to seal the aperture of the dropper when the base is releasably engaged with the dropper.

18. The sampling device of claim 17, wherein, adjacent the aperture, the dropper has a contoured outer surface and wherein the sealing surface is configured to contact and conform to the contoured outer surface of the dropper.

19. The sampling device of claim 17 or 18, wherein the sealing surface comprises a projection that covers or projects into the aperture of the dropper.

20. The sampling device of any one of claims 17 to 19, wherein the base comprises a cup and an insert located in the cup, the insert comprising the sealing surface.

21. The sampling device of any one of claims 17 to 19, wherein the base comprises a gasket, the gasket comprising the sealing surface.

22. The sampling device of any one of the preceding claims, wherein the dropper comprises a protrusion against which the absorbent portion moves when received in the internal chamber of the dropper to assist in release of sample from the absorbent portion.

23. The sampling device of any one of the preceding claims, wherein a fluid is provided in the internal chamber of the dropper.

24. The sampling device of any one of the preceding claims, wherein a reagent is provided in the internal chamber of the dropper.

25. The sampling device of claim 24, wherein the reagent comprises particles configured to label a target analyte in the sample.

26. The sampling device of any one of the preceding claims where in the sample is a nasal sample.

27. The sampling device of claim 26, wherein the sample collector is configured to be at least partially inserted into a nasal cavity to collect the sample.

28. A method of collecting a sample and dispensing a portion of the sample to a test device, the method comprising:
using an absorbent portion of a sample collector to collect a sample;
after collecting the sample, securing the sample collector to a dropper of a receiving section, wherein the receiving section comprises the dropper and a base that is releasably engaged with, and that at least partially houses, the dropper;
after securing the sample collector to the dropper, releasing the dropper from engagement with the base; and
dispensing a portion of the sample from an aperture of the dropper.

29. A sampling device for collecting and dispensing a sample, the sampling device comprising:
a sample collector comprising:
a cap; and
an insertion portion having a proximal end and a distal end, a proximal end of the insertion portion being connected to the cap and an absorbent portion being located at or adjacent the distal end of the insertion portion, the absorbent portion being configured to collect a sample;
a receiving section having an internal chamber, the sample collector being configured to be coupled to the receiving section after collection of the sample, wherein the cap at least partially closes the internal chamber and the absorbent portion of the sample collector is received in the internal chamber;
wherein, when the absorbent portion is received in the internal chamber, the absorbent portion interferes with an extraction surface of the receiving section to cause extraction of sample from the absorbent portion into the internal chamber.

30. The sampling device of claim 29, wherein the extraction surface is a surface of a wall that at least partially defines the internal chamber of the receiving section.

31. The sampling device of claim 29 or 30, wherein the extraction surface comprises at least part of a bottom surface of the internal chamber and/or at least part of a side surface of the internal chamber.

32. The sampling device of claim 29, 30 or 31, wherein the absorbent portion deforms when it interferes with the extraction surface.

33. The sampling device of claim 32, wherein the length of the insertion portion is such that, when absorbent portion is received in the internal chamber, the absorbent portion cannot be accommodated in the internal chamber without deforming.

34. The sampling device of claim 32 or 33, wherein the absorbent portion deforms by bending.

35. The sampling device of claim 34, wherein the absorbent portion bends by an angle of at least 90 degrees, at least 120 degree or at least 150 degrees.

36. The sampling device of any one of claims 32 to 35, wherein the absorbent portion deforms by bending back and pressing against itself.

37. The sampling device of any one of 32 to 36, wherein the absorbent portion is elongate and the absorbent portion bends at a central portion thereof.

38. The sampling device of claim 37, wherein at least half of a length of the absorbent portion is configured to interfere with the extraction surface.

39. The sampling device of any one of claims 29 to 38, wherein the absorbent portion comprises absorbent material surrounding a flexible rod portion.

40. The sampling device of any one of claims 29 to 39, wherein securing the sample collector to the receiving section after collection of the sample forms a dropper from which sample is dispensable.

41. The sampling device of claim 40, wherein the receiving section comprises flexible side walls pressable by a user of the device to cause dispensing of the sample from the internal chamber.

42. The sampling device of any one of claims 29 to 41, wherein the cap comprises at least one aperture through which sample can be dispensed from the sampling device.

43. The sampling device of claim 42, wherein the cap comprises a lid that is movable between an open and closed position to selectively seal the aperture.

44. The sampling device of claim 43, wherein a fluid path is provided through the insertion portion body at a proximal region of the insertion portion.

45. The sampling device of claim 44, wherein the proximal region of the insertion portion comprises one or more openings adjacent the aperture.

46. The sampling device of claim 45, wherein the insertion portion comprises spaced apart legs that connect the insertion portion to the cap at connection positions that are spaced around the aperture.

47. The sampling device of any one of claims 29 to 46, wherein the sample collector is secured to the receiving section by a screw-fitting.

48. The sampling device of any one of claims 1 to 27 or 29 to 47, comprising a desiccant housing.

49. The sampling device of claim 48, wherein the receiving section comprises a base and the desiccant housing is comprised in the base.

50. The sampling device of claim 48 or 49, wherein the desiccant housing comprises one or more windows.

51. Sample collector apparatus comprising:
a sample collector comprising:
a handle; and
an insertion portion having a proximal end and a distal end, a proximal end of the insertion portion being connected to the handle and an absorbent portion being located at or adjacent the distal end of the insertion portion;
wherein the sample collector is configured to collect a sample by inserting a length of the insertion portion including the absorbent portion into a body cavity; and
a length adapter configured to be coupled to the sample collector to adjust a maximum insertion depth of the absorbent portion into the cavity.

52. The apparatus of claim 51, wherein the length adapter is configured to increase an effective diameter of a region of the insertion portion.

53. The apparatus of claim 51 or 52, wherein the length adapter is configured to be releasably coupled to the sample collector.

54. The apparatus of claim 51, 52 or 53, wherein the length adapter is configured to be releasably coupled to a proximal region of the insertion portion.

55. The apparatus of any one of claims 51 to 54, wherein the length adapter is configured to be coupled to the sample collector via an interference or friction fit.

56. The apparatus of any one of claims 51 to 55, wherein the length adapter is substantially annular.

57. The apparatus of any one of claims 51 to 56, wherein the length adapter has inner side walls and outer side walls, the inner side walls defining a central opening through which the insertion portion is extended when the length adapter is coupled to the sample collector.

58. The apparatus of claim 57, wherein the length adapter has a split in the inner and outer side walls.

59. The apparatus of claim 57 or 58, wherein the central opening has an outer diameter as defined by the inner side walls that substantially corresponds to the diameter of an outer surface of part of the insertion portion.

60. The apparatus of claim 57, 58 or 59, wherein the outer side walls have a diameter that substantially corresponds to a diameter of an outer surface of the handle.

61. The apparatus of any one of claims 57 to 60, wherein the outer side walls have a diameter that is greater than a diameter of an opening to the body cavity.

62. The apparatus of any one of claims 51 to 61, wherein the handle is a cap and the sample collector is configured to be coupled to a receiving section comprising an internal chamber, wherein the cap at least partially closes the internal chamber and the absorbent portion of the sample collector is received in the internal chamber.

63. A sampling device comprising:
a sample collector to collect a sample;
a receiving section, the sample collector being configured to be coupled to the receiving section after collection of the sample, the receiving section comprising a desiccant housing containing a desiccant.

64. The sampling device of claim 63, wherein the receiving section comprises a base and the desiccant housing is comprised in the base.

65. The sampling device of claim 63 or 64, wherein the desiccant housing comprises one or more windows.

66. The sampling device of any one of claims 63 to 65 wherein the receiving section comprises an internal chamber that receives an absorbent portion of the sample collector when the sample collector is coupled to the receiving section.

67. The sampling device of claim 66, wherein the receiving section comprises one or more reagents coated on or in a surface of the internal chamber or other item located in the internal chamber.
